(19) United States
(12) Patent Application Publication
(10) Pub. No.: US 2026/0157882 A1
(43) Pub. Date: Jun. 11, 2026

(54) TILT-RETRACTION EYE DROP DISPENSER, SYSTEM AND METHOD

(71) Applicant: Andrew Peter Davis, Bellevue, WA (US)

(72) Inventor: Andrew Peter Davis, Bellevue, WA (US)

(21) Appl. No.: 19/412,799

(22) Filed: Dec. 8, 2025

Related U.S. Application Data
(60) Provisional application No. 63/729,500, filed on Dec. 9, 2024.

(51) Int. Cl. A61F 9/00; A61M 35/00; B65D 47/18

(52) U.S. Cl. CPC A61F 9/0026; A61M 35/003; B65D 47/18; B65D 2203/12

(57) ABSTRACT

A device and method for dispensing an eye drop using a housing supporting the eye drop bottle and an electronic or mechanical sensor that senses when the nozzle of the eye drop bottle is aligned with the eye such that a drop of fluid can be dispensed toward a conjunctival target of the eye and generates an audible, visual, or tactile alert when aligned. A bottle compression assembly may be used to more easily dispense the fluid. The method includes positioning a housing supporting the eye drop bottle in a substantially upright position relative to the eye, retracting a lower lid of the eye to position the nozzle relative to a lid margin of the eye, tilting the housing until an alert is generated indicating that the nozzle is aligned with the eye, and compressing the eye drop bottle to dispense at least a drop of fluid towards the eye.

### PRIORITY CLAIM

This application claims the benefit of priority from US Provisional Ser. No. 63/729,500 , filed Dec. 9, 2024, the contents of which are incorporated herein by reference.

### BACKGROUND OF THE INVENTION

The self-administration of eye drops can be particularly challenging. The elderly can have difficulty squeezing a stiff bottle, especially if they have arthritis muscle weakness. Holding the bottle steady near the eye can be difficult if there is any type of tremor. Using a mirror can be difficult, especially if there is visual impairment or difficulty in getting close enough to a mirror. There is significant wasting of the eye drops due to missing the eye, and then having to put in multiple drops to ensure that at least one drop got in. Drops that miss the eye and land on the skin can cause significant irritation of the skin. Holding the bottle, holding the eye open, positioning a mirror, or squeezing a stiff bottle can be a daunting task for many individuals and their families.

There are many products on the market to help make giving eyedrops easier. Many involve a bottle holder to make it easier to squeeze the bottle and a guide to hold the bottle above the eye. Other products let the bottle rest on the bridge of the nose. A solution to these issues becomes apparent when observing how eye doctors and their staff administer eye drops to a multitude of patients each day. Their specific technique makes this task easy for both the patient as well as for the doctor. The technique involves the following steps: The patient is first asked to tilt their head back. The doctor then holds the eye drop bottle like a pencil and tilts it downwards towards the eye at about the same angle that the head is tilted. Next, the doctor uses their third or fourth finger of the same hand to retract the lower lid downwards. The doctor then squeezes the bottle to administer the drop into the exposed lower conjunctiva and cul-de-sac. The invention described here lets the patient administer their own drops with a similar technique and at the same time facilitates holding the bottle steady as well as squeezing a stiff bottle.

### SUMMARY OF THE INVENTION

The present is in improved device and method for delivering liquid from an eye drop bottle to an eye. In one embodiment, a housing is configured to support a standard commercial eye drop bottle, the housing supports a sensor configured to sense when the nozzle of the eye drop bottle is aligned with the eye such that at least one drop of fluid from the eye drop bottle can be dispensed toward a conjunctival target of the eye, and further to generate an audible, visual, or tactile alert when the nozzle of the eye drop bottle is aligned. The device may include a separate alert generator in communication with the sensor configured to generate an audible, visual, or tactile alert generator when the nozzle of the eye drop bottle is aligned.

In an alternative embodiment, the device may include a bottle compression assembly configured discharge a drop of fluid from the eye drop bottle. The bottle compression assembly may further include a handle and a squeeze lever used to generate compression of the eye drop bottle, and also the handle may be configured to orient the device with respect to the eye while the squeeze lever used to generate compression of the eye drop bottle.

In a different embodiment, the device may include a retractor configured to retract a lower lid of the eye before the drop of the fluid is dispensed towards the eye, and the retractor may be configured to be adjusted in at least one of position, length or orientation.

In certain embodiments, the device sensor is triggered when the device is tilted at approximately 45 degrees, although the sensor may be configurable to modify when the resulting alert is generated when the nozzle of the eye drop bottle is aligned.

In yet alternative embodiments, the device includes an eye drop bottle holder that orients the nozzle of the eye drop bottle downwards. The eye drop bottle holder may be configured to support eye drop bottles of different sizes without changing the orientation of the nozzle of the eye drop bottle.

In alternative embodiments, the sensor consists of a ball, bell or similar generally spherical element configured to move along a contained path and generate an alert when the nozzle of the eye drop bottle is aligned with the eye such that at least one drop of fluid from the eye drop bottle can be dispensed toward a conjunctival target of the eye.

In yet an alternative embodiment, a device for delivering liquid from an eye drop bottle having a nozzle to an eye includes a bottle compression assembly configured discharge a drop of fluid from the eye drop bottle and a sensor configured to sense when the nozzle of the eye drop bottle is aligned with the eye such that at least one drop of fluid from the eye drop bottle can be dispensed toward a conjunctival target of the eye, and further to generate an audible, visual, or tactile alert when the nozzle of the eye drop bottle is aligned. The device may include a separate alert generator in communication with the sensor configured to generate an audible, visual, or tactile alert generator when the nozzle of the eye drop bottle is aligned. The bottle compression assembly may further include a handle and a squeeze lever used to generate compression of the eye drop bottle, and also the handle may be configured to orient the device with respect to the eye while the squeeze lever used to generate compression of the eye drop bottle. The device may include a retractor configured to retract a lower lid of the eye before the drop of the fluid is dispensed towards the eye, and the retractor may be configured to be adjusted in at least one of position, length or orientation. The device sensor may be triggered when the device is tilted at approximately 45 degrees, although the sensor may be configurable to modify when the resulting alert is generated when the nozzle of the eye drop bottle is aligned. The device may include an eye drop bottle holder that orients the nozzle of the eye drop bottle downwards, which also may be configured to support eye drop bottles of different sizes without changing the orientation of the nozzle of the eye drop bottle. The sensor may consist of at least one of a ball or bell configured to move along a contained path and generate an alert when the nozzle of the eye drop bottle is aligned with the eye such that at least one drop of fluid from the eye drop bottle can be dispensed toward a conjunctival target of the eye.

A method for delivering liquid from an eye drop bottle having a nozzle to an eye includes the steps of positioning a housing supporting the eye drop bottle in a substantially upright position relative to the eye, retracting a lower lid of the eye to position the nozzle of the eye drop bottle relative to a lid margin of the eye, tilting the housing supporting the eye drop bottle until an alert is generated indicating that the nozzle of the eye drop bottle is aligned with the eye, and compressing the eye drop bottle to dispense at least a drop of fluid towards the eye. The method may be adapted such that the alert is generated when the nozzle of the eye drop bottle is aligned with the eye such that at least one drop of fluid from the eye drop bottle can be dispensed toward a conjunctival target of the eye. The alert may be an audible, visual, or tactile alert. The step of compressing the eye drop bottle to dispense at least a drop of fluid towards the eye may be accomplished using a bottle compression assembly comprising a handle and a squeeze lever used to generate compression of the eye drop bottle. The sensor may consist of at least one of a ball or bell configured to move along a contained path and generate an alert when the nozzle of the eye drop bottle is aligned with the eye such that at least one drop of fluid from the eye drop bottle can be dispensed toward a conjunctival target of the eye.

### BRIEF DESCRIPTION OF THE DRAWINGS

Preferred and alternative examples of the present invention are described in detail below with reference to the following drawings.

FIG. 1 shows a tilt-retraction eye drop dispenser according to an embodiment of the present invention.

FIG. 2 shows a cutaway view of the tilt-retraction eye drop dispenser according to an embodiment of the present invention.

FIGS. 3A-3B show an exemplary bottle compression assembly of an embodiment of the present invention.

FIGS. 4A-4B show an alternative embodiment of the present invention having a mechanical tilt alarm.

FIGS. 5A-5D show the operation of the tilt-retraction eye drop dispenser of FIGS. 4A-4B.

FIG. 6 shows an enlarged view of the tilt-retraction eye drop dispenser of FIGS. 4A-4B illustrating the relative position of an eye in dispenser operation.

FIG. 7 shows a flowchart detailing one method of using the present invention.

FIG. 8 shows the tilt-retraction eye drop dispenser of FIGS. 4A-4B, further incorporating an adaptor configured to facilitate compression of an eye drop bottle (not shown).

FIGS. 9A-9C show an alternative tilt-retraction eye drop dispenser in accordance with the present invention without use of a bottle compression assembly.

### DETAILED DESCRIPTION OF THE PREFERRED EMBODIMENT

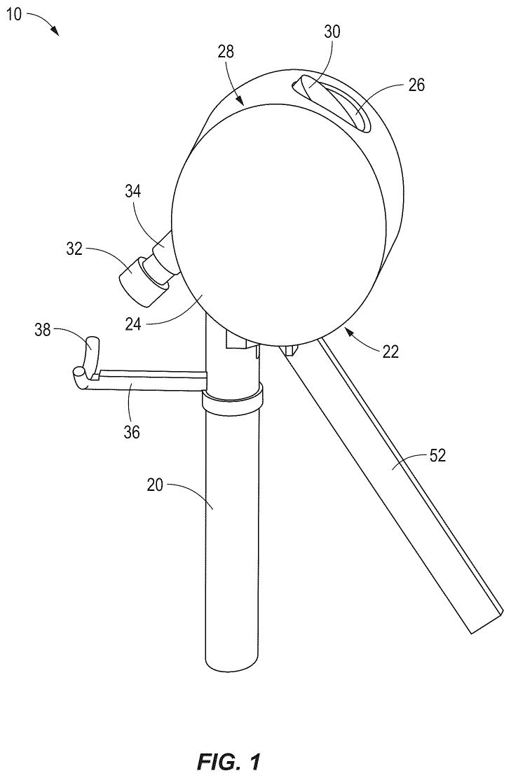

A tilt-retraction eye drop dispenser 10 to be used with an eye (not shown) is described with reference to FIGS. 1-3. Dispenser 10 includes a substantially vertical handle 20 attached to a lower part 22 of a housing 24. The housing 24 includes an opening 26 in an upper part 28 of the housing, the opening configured to accommodate insertion of a standard eye drop bottle 30, or any type of container used to dispense a liquid eye drop solution that includes a nozzle 31 having a nozzle aperture (not shown) from which liquid eye drops are dispensed. The eye drop bottle 30 is preferably inserted into the housing 24 in a removable fashion, for example by friction fit, screwed into threads on the nozzle or bottle corresponding to threads in the nozzle, or the like, but may also be secured in a non-removable fashion, for example by glue or the like. It should be noted that the eye drop bottle (or container), as described herein, need not be fully or even partially vertical in operation. Rather, it need only be oriented downward sufficient that at least a portion of the liquid retained therein accumulates in the nozzle for transmission to the compression chamber or dispenser, depending on the embodiment. A bottle cap 32 is configured to cover a nozzle holder 34 extending from the housing 24 and configured to accommodate a nozzle 31 of the eye drop bottle 30. In a preferred embodiment, a retractor assembly comprising a retractor 36 configured to retract a lower eye lid (not shown) extends from the housing 24 or the handle 20 and a retractor pad 38. The retractor assembly and its components can be positioned at different locations with respect to the housing or bottle compression assembly, and configured to be adjusted in terms of size, length or orientation depending on the needs of the user. Inclusion of the retractor 36 and/or retractor pad 38 is only one variation of the present invention. It will be appreciated that any alternative embodiment may not include the either or both elements, which are optional features.

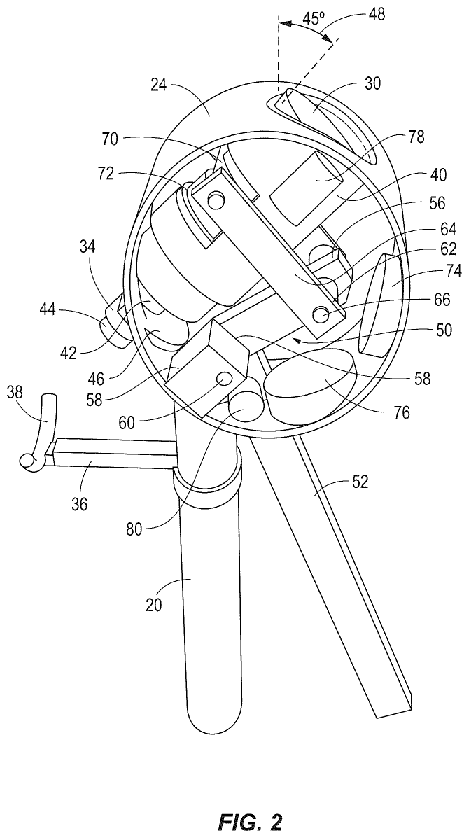

As shown in more detail with reference to FIGS. 1 and 2, the dispenser 10 includes a bottle holder 40, preferably supported within or by the housing 24, configured to secure the eye drop bottle 30 upon insertion via the opening 26. The bottle holder 40 facilitates fluidic connection between the nozzle 31 of the eye drop bottle 30 and a dispenser aperture 44 connected to the nozzle holder 34, preferably via a tube or equivalent structure 42 running therebetween. A nozzle platform 46 is configured to support the eye drop bottle 30 within the housing 24. The bottle holder 40 is preferably configured to support the eye drop bottle 30 at an angle of 45 degrees, as shown within the housing range 48.

The dispenser 10 further includes a bottle compression assembly 50 supported by the housing 24. The compression assembly 50 includes a lever arm 52 proximate to the handle 20 that preferably enters the housing 24 via the housing lower part 22. The lever arm 52 is L-shaped, with the short end of the L-shaped arm having an angle point end 54 connected to the long end of the L-shaped arm and an opposite distal end 56. The level arm 52 is pivotably connected at its angle point end 54 to a stop 58 via a stop axle 60 and at its distal end 56 to a first end 62 of a compressor lever 64 via a compression axle 66. A second end 68 of the compressor lever 64 is connected to a compressor 70, which is configured to compress the eye drop bottle 30, in this embodiment through a bottle holder opening 72, which compressor is configured to force at least a drop of fluid out of the eye drop bottle when the lever arm 52 is operated or squeezed towards the handle 20. The use of a bottle compression assembly and compressor as described herein provide a mechanical advantage in dispensing liquid from an eye drop bottle as it requires less force to compress the bottle.

The dispenser 10 further includes a battery 74, an alert such as a piezo buzzer or other noise maker 76, and a vertical sensor 78 configured to sense tilting of the dispenser in a substantially vertical direction, in a preferred embodiment about 45 degrees, and a sideways tilt sensor 80 configured to sense tilting in a substantially horizontal or sideways direction. Means for generating various types of alerts (or as used herein alert generators) may include but are not limited to the following:
Audible Alerts: buzzers, or electronic devices emitting a repetitive buzzing or beeping sound; horns, such as electro-mechanical or electronic units providing singular or continuous tones; sirens; bells; chimes; electronic sounders; or mechanical devices such as balls, marbles, clickers, or the like.
Visual Alerts: LED lights that may be wired or wireless with customizable colors (e.g., red, green, blue, amber); strobe lights, steady indicator lights (VIDs); incandescent/halogen lights; laser diodes; or the like.
Tactile Alerts: In addition to certain audible alerts that also generate tactile or haptic notification, devices such as vibrating pads or other structural components, or the like.

As used herein, the sensor can be any electronic or mechanical device configured to sense orientation of an attached structure within a predetermined range, for example a tilt sensor that senses vertical or horizontal movement of a structure. It will be appreciated that both sensors needs not be included, and only a single tilt sensor could be used. Also, rather than a separate alert, either or both sensors may include a means to provide an alert when predetermined conditions are met, for example to generate an audible, visual or tactile (or combination thereof) alert, namely, anything that may be used to get the attention of a user. As used herein, regardless of the type of sensor or whether the sensor is configured to generate an alert or connected to a separate device to generate an alert, the sensor is configured to sense when the eye drop bottle is aligned with the eye (or designed target associated therewith) such that at least one drop of fluid can be dispensed from the eye drop bottle towards the eye, preferably with the aid of gravity.

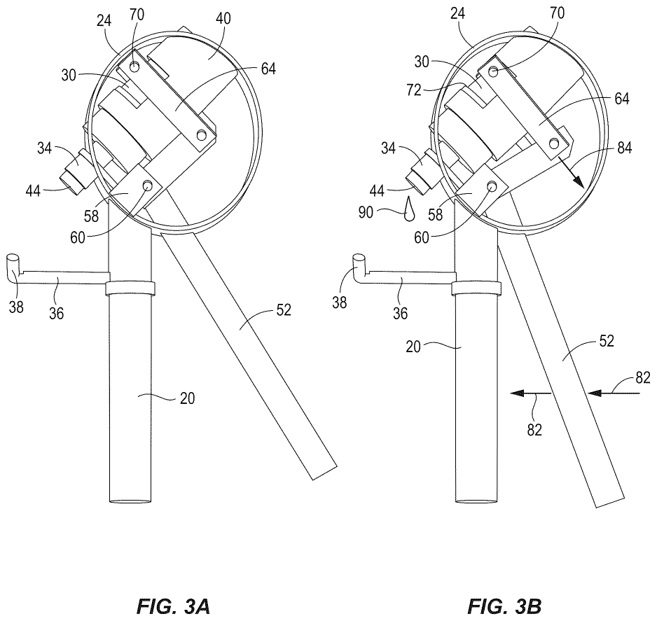

The operation of the embodiment of the tilt-retraction eye drop dispenser 10 shown in FIGS. 1-2 is illustrated further with reference to FIGS. 3A-3B. In FIG. 3A, the lever arm 52 is positioned proximate to the handle 20 so that it can easily be grasped and compressed while holding the handle. In FIG. 3B, the lever arm 52 is squeezed or compressed towards the handle 20 generally in the direction shown by arrows 82, which causes the lever arm 52 to rotate on stop axle 62, thereby causing the downward movement of the compressor lever 56 generally in the direction shown by arrow 84, which in turn pulls the compressor 70 downward in the same direction and compresses the eye drop bottle 30 to discharge an eye drop 90.

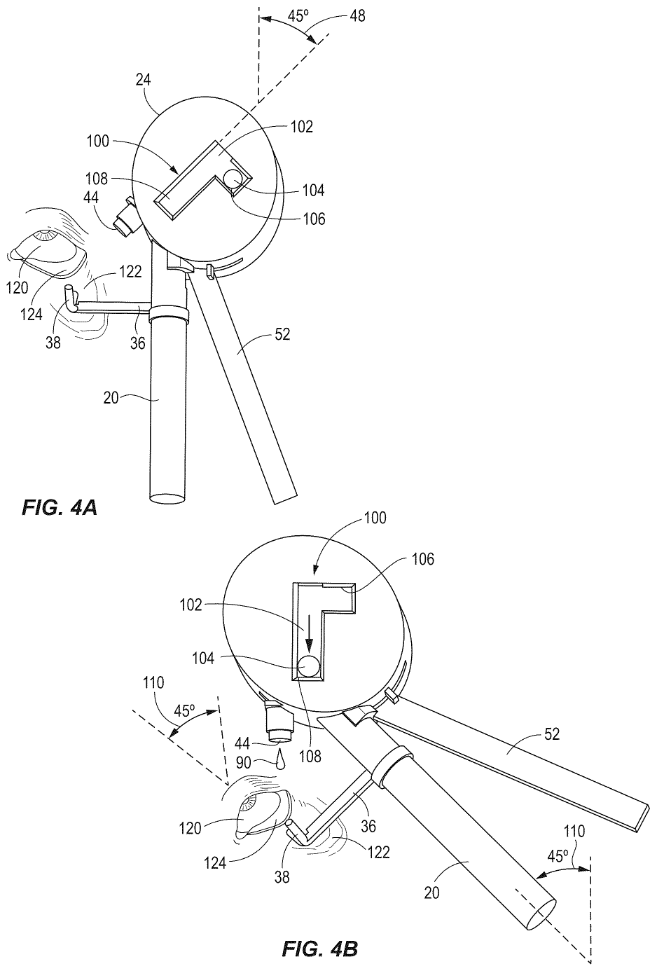

An alternative embodiment of the tilt-retraction eye drop dispenser 10 using a mechanical tilt alert is shown more specifically with reference to FIGS. 4-6. In this embodiment, the tilt alert is mechanical instead of electronic, avoiding the need for a battery, buzzer, or electronic sensor. FIG. 4A shows a mechanical tilt alert assembly 100 including an L-shaped channel or tunnel 102 supported by the housing 24 at preferably a 45 degree angle, as shown by housing range 48. The assembly 100 is configured so that when the housing 24 is upright, a generally or approximately round or spherical ball or bell 104 (preferably configured to roll) movably contained within the tunnel 102 is resting in the short arm 106 of the tunnel 106 As shown in FIG. 4B, when the housing 24 and the eye 120 and lower eyelid 122 are tilted to 45 degrees as shown generally by eye range 110, the ball 104 rolls down the short arm 106 and then drops to the bottom of the long arm 108, causing a sound to alert a user to stop tilting their head and discharge the single eye drop 90 of fluid. FIGS. 4A-4B shown use of a retractor 36 and retractor pad 48 to retract the lower eyelid 122. As described herein, inclusion of the retractor 36 and/or retractor pad 38 is only one variation of the present invention. It will be appreciated that any alternative embodiment may not include the retractor assembly, which is an optional feature. Instead, a user's finger or fingers may be used to retract the lower eyelid 122 downwards so that the dispenser aperture is aimed above the lower eyelid 122.

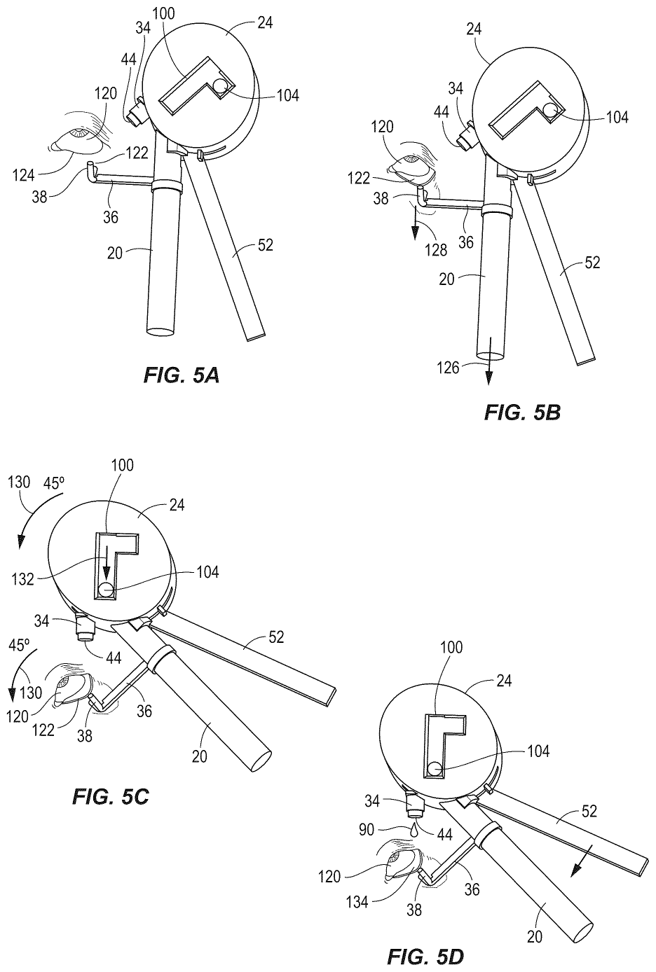

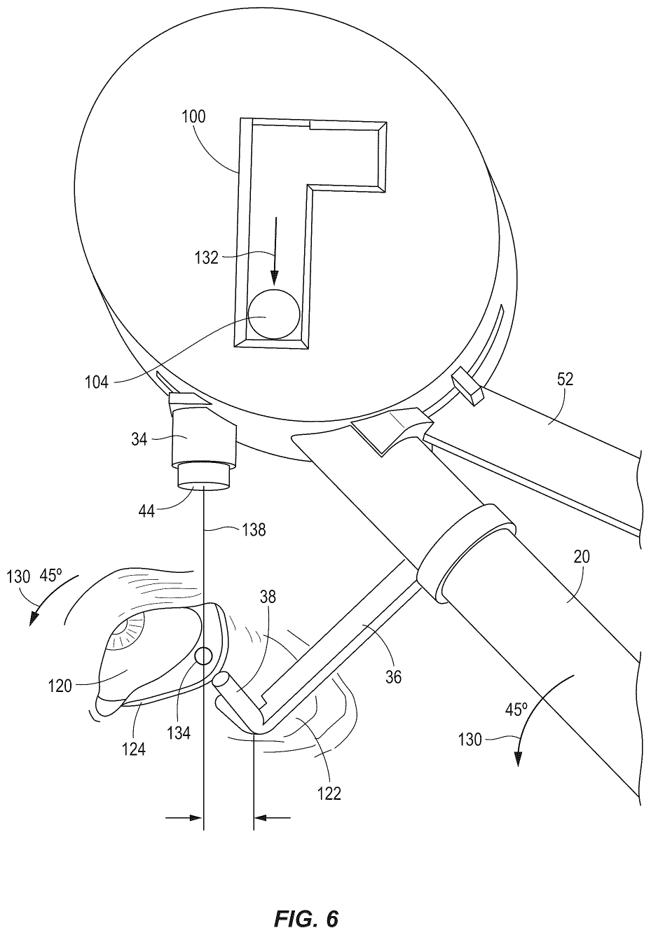

The operation of the tilt-retraction eye drop dispenser 10 is illustrated further with reference to FIGS. 5A-5D and 6. The method of operation is substantially the same regardless of whether electronic or mechanical alert is incorporated into a particular embodiment. The operation is shown incorporating the retractor 36 and/or retractor pad 38, but as noted the operation may be performed instead with a user's finger or fingers used to retract the lower eyelid 122 to discharge the eye drop 90.

In FIG. 5A, the tilt-retraction eye drop dispenser 10 is positioned with the handle 20 in a substantially upright position in front of the eye 120, with the retractor pad 38 of the retractor 36 engaging the lower lid 122 just below the lid margin 124. The alert ball 104 remains in a non-alert condition in the tunnel short arm, or in alternative embodiment using an electric alert, the alert remains in a non-alert condition. In FIG. 5B, the dispenser 10 is pulled downward in generally the direction shown by arrow 126, which in turn pulls the retractor pad 38 to retract the lower eyelid 122 generally in the direction shown by arrow 128. The alert ball 104 remains in a non-alert condition in the tunnel short arm, or in alternative embodiment using an electric alert, the alert remains in a non-alert condition.

While maintaining this position of the dispenser 10 relative to the eye 120, as shown in FIG. 5C, the user slowly and preferably simultaneously tilts the dispenser 10 and their head (not shown) and corresponding eye 120 and lower eyelid 122 along an arc as shown generally by arrows 130. At an approximate 45 degree tilt, the alert ball 104 moves along the L-shaped tunnel from the tunnel short arm 106 into the tunnel long arm 108 generally in the direction shown by arrow 132. In alternative embodiment using an electric alert, the alert remains in a non-alert condition until at an approximate 45 degree tilt. At that point, the alert sensor (whether mechanical ball drop or electronic sensor) is triggered, indicating that the orientation of the dispenser 10 relative to the eye 120 is optimal—or within an acceptable range—to align the dispenser aperture 44 with the eye 120 above the lid margin 124 and facilitate dispensing the eye drop 90, at which point the user stops further movement. Note that experimentation and anecdotal observation has confirmed that an approximate 45 degree tilt provides an optimal orientation for dispensing the eye drop. It will be appreciated, however, that there is a range greater or less than 45 degrees that forms an acceptable range of tilt to provide adequate eye drop delivery, which may in part be based on variations in human eye location and orientation and user movement. It is understood that that alert sensor (whether mechanical or electronic) can be modified to enlarge or reduce this acceptable range, depending on user preference. For example, with the mechanical embodiment, the shape of the tunnel 102 can be modified from a right-angle L-shape to a different shape, which will change the point at which the ball moves from the non-alert to an alert condition. Once the dispenser 10 is aligned as desired with respect to the eye 120, as shown in FIG. 5D, the user operating the dispenser by squeezing the lever arm 52 towards the handle 20, which operates the bottle compression assembly 50 as described above to compress the eye drop bottle 30, which forces at least a drop of fluid out of the eye drop bottle 30 to dispense an eye drop 90 towards the eye 120, preferably to a conjunctival target 132, or the area of conjunctiva below the eye 120 and inside the lower eyelid 122, as well as the pocket between them.

FIG. 6 shows an enlarged schematic of the relative positions of the eye 120 and dispenser 10 when the tilt alarm (whether mechanical or electronic) sounds. When the dispenser 10 and user's head (not shown) tilted to approximately 45 degrees as shown generally by arrow 130, the ball 104 drops generally in the direction shown by arrow 132 and alerts the user to squeeze the lever arm 52 to dispense the eye drop 90. The nozzle 31 and corresponding dispenser aperture is substantially vertical since it is in the housing 24 at approximately an angle of 45 degrees, and the housing is tilted approximately 45 degrees. It will be appreciated that dispenser aperture is preferably either the opening of the nozzle 31 of the eye drop bottle 30 or other aperture in fluidic communication with the nozzle through which eye drop fluid travels from the eye drop bottle out of the dispenser 10. The nozzle 31 and the retractor pad 38 of the retractor 36 are preferably positioned so that a vertical axis 138 of the nozzle 31 passes in front of the retractor pad 38 by a sufficient clearance distance shown as the distance between the lines marked 138 to ensure that the vertical axis 136 intersects the conjunctival target area 134, which is exposed by the lower eyelid 122 being retracted, the head and eye 120 tilted back, and the user looking up. In this preferred embodiment, the clearance distance 138 is approximately 12 mm, but that distance may vary based on factors including user head and eye characteristics and dispenser and retractor size and orientation.

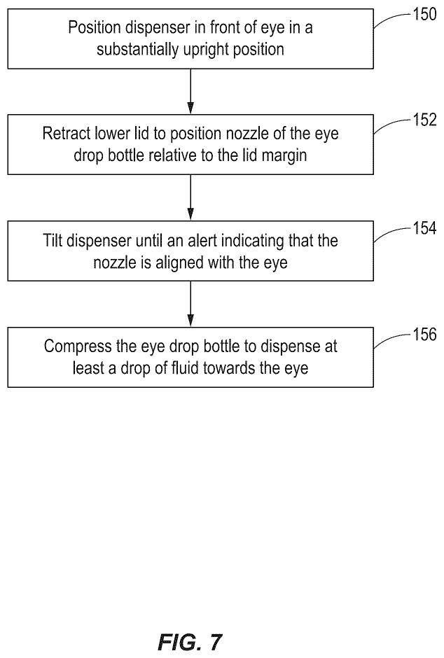

FIG. 7 detailed a preferred methodology of operation of the tilt-retraction eye drop dispenser 10 of present invention. At block 150, the dispenser 10 is positioned with the handle 20 in a substantially upright position in front of the eye 120. At block 152, the lower lid 122 is retracted to position the dispenser aperture 44 above the lid margin 124. In one embodiment, the retractor pad 38 of the retractor 36 engages the lower lid 122 just below the lid margin 124 to retract the lower lid and position the dispenser aperture 44 above the lid margin. In an alternative embodiment where the present invention does not include a retractor, a user's finger(s) may be used to retract the lower lid and position the dispenser aperture above the lid margin. At block 154, the dispenser and user's head and corresponding eye 120 are tilted back until an alert sensor (whether mechanical ball drop or electronic sensor) is triggered, indicating that the orientation of the dispenser 10 relative to the eye 120 is optimal—or within an acceptable range—to align the dispenser aperture 44 with the eye 120 above the lid margin 124 and facilitate dispensing the eye drop 90, at which point the user stops further movement. It should be appreciated that the actions of block 152 could be perform simultaneous with or part way through the actions of block 154. In other words, the lower lid may be retracted during the actions of tilting the dispense and head back in block 154, or even after the alert is generated in block 154, but before commencement of block 156. At block 156, the dispenser is operated by squeezing the lever arm 52 towards the handle 20, which operates the bottle compression assembly 50 to compress the eye drop bottle 30 and force at least a drop of fluid out of the eye drop bottle 30 to dispense an eye drop 90 towards the eye 120, preferably to the conjunctival target 132, or the area of conjunctiva below the eye 120 and inside the lower eyelid 122, as well as the pocket between them.

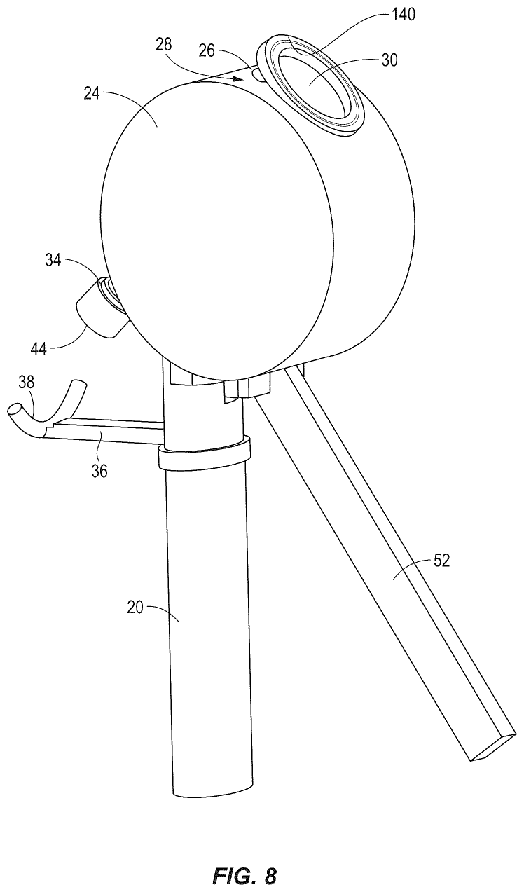

Commercially available eye drop bottles may vary in size. FIG. 8 shows an alternative embodiment including an bottle adapter 140 configured to insert into the opening 26 in the upper part 28 of the housing 24. The adaptor consists of a tube or sleeve having an inner diameter configured to adapt to the variable diameter of non-standard eye drop bottle, for example smaller bottles, to allow the bottle to be retained within the bottle holder 40 and configured to be aligned with and subject to operation by the compression assembly. The adaptor may optionally include an opening (not shown) configured to align with the opening 26 in the bottle holder 40 to allow the compressor 70 to compress against the eye drop bottle 30 through the adaptor.

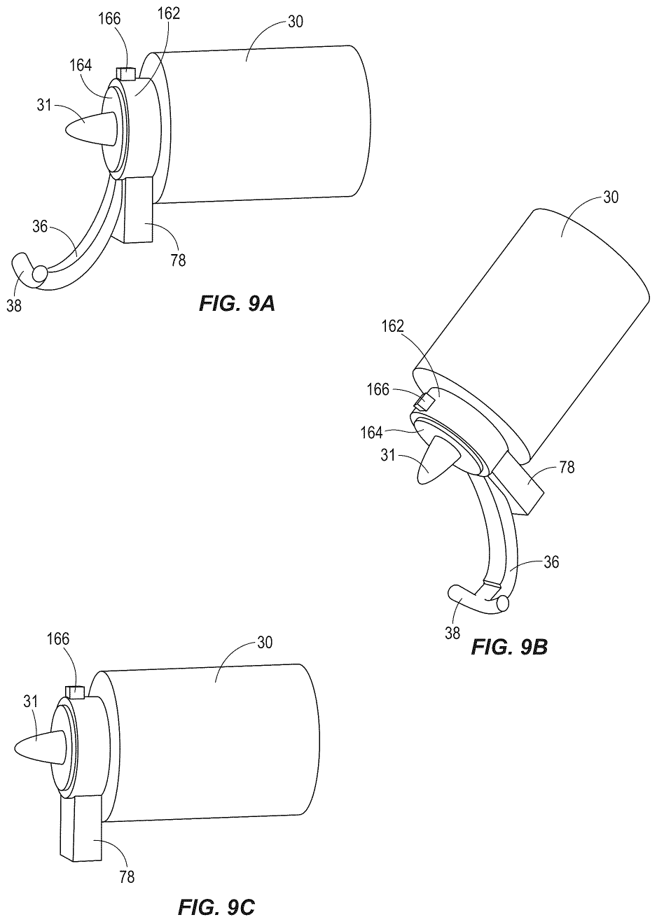

An alternative embodiment of the tilt-retraction eye drop dispenser 10 of present invention that does not incorporate a compression assembly is shown with reference to FIGS. 9A-9C. In this embodiment, a housing 160 consists of a bottle ring 162 that screws or otherwise removably or permanently connects onto the eye drop bottle 30, in this embodiment screwed on via the eye drop bottle neck 164. The electronic tilt sensor 78 is connected to the housing 160. Optional retractor 36 and retractor pad 38 are shown in this embodiment to retract the lower eyelid 122, but it will be appreciated that neither need be included, as a user's finger(s) may be used for the same purpose, as shown in FIG. 9C. An LED or other visual alert 166 is connected to the housing 160, preferably on an upper part of the bottle ring 162 where it is visible to one of both of the user's eyes. In FIG. 9B, the patient grasps the eye drop bottle 30, engages and retracts the lower lid downwards (either using the retractor 36 and retractor pad 38 or finger(s)), and then tilts their head back until the tilt sensor 78 is triggered and makes a sound and lights up the LED, signaling that it is in proper position to squeeze the eye drop bottle 30 and administer at least one drop 90. In alternative embodiments, a sensor having only a single alert, either audible or visual, may be used, rather than both.

While the preferred embodiment of the invention has been illustrated and described, as noted above, many changes can be made without departing from the spirit and scope of the invention. For example, in alternative embodiments, the bottle compression assembly may be configured to independent support other elements of the present invention, such as the nozzle holder, nozzle platform, optional retractor and retractor pad, electronic alert components such as a battery, sensor(s), alert devices (if separate from the sensor(s)), and mechanical components such as including a tunnel and ball. In certain embodiments, the sensor may include an alert, while in other embodiments a separate device may produce an alert, and in either case the alert may be audible (e.g., buzzer, noise maker, mechanical sound generator such as the drop of a ball or marble), visual (e.g., an LED or other light visual to the user), or tactile (e.g., a device to create a pulse or vibration felt by the user). Accordingly, the scope of the invention is not limited by the disclosure of the preferred embodiment. Instead, the invention should be determined entirely by reference to the claims that follow.

The embodiments of the invention in which an exclusive property or privilege is claimed are defined as follows:

1. A device for delivering liquid from an eye drop bottle having a nozzle to an eye, comprising:
a housing configured to support the eye drop bottle;
a sensor supported by the housing and configured to:
sense when the nozzle of the eye drop bottle is aligned with the eye such that at least one drop of fluid from the eye drop bottle can be dispensed toward a conjunctival target of the eye; and
generate at least one of an audible, visual, or tactile alert when the nozzle of the eye drop bottle is aligned.

2. The device of claim 1, wherein an alert generator in communication with the sensor is configured to generate at least one of an audible, visual, or tactile alert generator when the nozzle of the eye drop bottle is aligned.

3. The device of claim 1, further comprising a bottle compression assembly configured discharge a drop of fluid from the eye drop bottle.

4. The device of claim 3, wherein the bottle compression assembly comprises a handle and a squeeze lever used to generate compression of the eye drop bottle.

5. The device of claim 3, wherein the handle is configured to orient the device with respect to the eye while the squeeze lever used to generate compression of the eye drop bottle.

6. The device of claim 1, further comprising a retractor configured to retract a lower lid of the eye before the drop of the fluid is dispensed towards the eye.

7. The device of claim 6, wherein the retractor is configured to be adjusted in at least one of position, length or orientation.

8. The device of claim 1, wherein the alert is triggered when the device is tilted at approximately 45 degrees.

9. The device of claim 1, further comprising an eye drop bottle holder that orients the nozzle of the eye drop bottle downwards.

10. The device of claim 1, further comprising an eye drop bottle holder configured to support eye drop bottles of different sizes without changing the orientation of the nozzle of the eye drop bottle.

11. The device of claim 1, wherein the sensor is configurable to modify when the alert is generated when the nozzle of the eye drop bottle is aligned.

12. The device of claim 1, wherein the sensor consists of at least one of a ball or bell configured to move along a contained path and generate an alert when the nozzle of the eye drop bottle is aligned with the eye such that at least one drop of fluid from the eye drop bottle can be dispensed toward a conjunctival target of the eye.

13. A device for delivering liquid from an eye drop bottle having a nozzle to an eye, comprising:
a bottle compression assembly configured discharge a drop of fluid from the eye drop bottle; and
a sensor supported by the bottle compression assembly and configured to:
sense when the nozzle of the eye drop bottle is aligned with the eye such that at least one drop of fluid from the eye drop bottle can be dispensed toward a conjunctival target of the eye; and
generate at least one of an audible, visual, or tactile alert when the nozzle of the eye drop bottle is aligned.

14. The device of claim 13, wherein an alert generator in communication with the sensor is configured to generate at least one of an audible, visual, or tactile alert generator when the nozzle of the eye drop bottle is aligned.

15. The device of claim 13, wherein the bottle compression assembly comprises a handle and a squeeze lever used to generate compression of the eye drop bottle.

16. The device of claim 15, wherein the handle is configured to orient the device with respect to the eye while the squeeze lever used to generate compression of the eye drop bottle.

17. The device of claim 13, further comprising a retractor configured to retract a lower lid of the eye before the drop of the fluid is dispensed towards the eye.

18. The device of claim 17, wherein the retractor is configured to be adjusted in at least one of position, length or orientation.

19. The device of claim 13, wherein the alert is triggered when the device is tilted at approximately 45 degrees.

20. The device of claim 13, further comprising an eye drop bottle holder that orients the nozzle of the eye drop bottle downwards.

21. The device of claim 13, further comprising an eye drop bottle holder configured to support eye drop bottles of different sizes without changing the orientation of the nozzle of the eye drop bottle.

22. The device of claim 13, wherein the sensor is configurable to modify when the alert is generated when the nozzle of the eye drop bottle is aligned.

23. The device of claim 13, wherein the sensor consists of at least one of a ball or bell configured to move along a contained path and generate an alert when the nozzle of the eye drop bottle is aligned with the eye such that at least one drop of fluid from the eye drop bottle can be dispensed toward a conjunctival target of the eye.

24. A method for delivering liquid from an eye drop bottle having a nozzle to an eye, comprising:
positioning a housing supporting the eye drop bottle in a substantially upright position relative to the eye;
retracting a lower lid of the eye to position the nozzle of the eye drop bottle relative to a lid margin of the eye;
tilting the housing supporting the eye drop bottle until an alert is generated indicating that the nozzle of the eye drop bottle is aligned with the eye; and
compressing the eye drop bottle to dispense at least a drop of fluid towards the eye.

25. The method of claim 24, wherein the alert is generated when the nozzle of the eye drop bottle is aligned with the eye such that at least one drop of fluid from the eye drop bottle can be dispensed toward a conjunctival target of the eye.

26. The method of claim 24, wherein the alert is at least one of an audible, visual, or tactile alert.

27. The method of claim 24, wherein compressing the eye drop bottle to dispense at least a drop of fluid towards the eye is accomplished using a bottle compression assembly comprising a handle and a squeeze lever used to generate compression of the eye drop bottle.

28. The method of claim 24, wherein the sensor consists of at least one of a ball or bell configured to move along a contained path and generate an alert when the nozzle of the eye drop bottle is aligned with the eye such that at least one drop of fluid from the eye drop bottle can be dispensed toward a conjunctival target of the eye.
